(19) United States
(12) Patent Application Publication
(10) Pub. No.: US 2017/0330438 A1
(43) Pub. Date: Nov. 16, 2017

(54) AUTONOMOUS LIFE MONITOR SYSTEM

(71) Applicant: iBeat, Inc., San Francisco, CA (US)

(72) Inventors: Ryan Howard, San Francisco, CA (US); Lawrence Stone, San Francisco, CA (US); Brian Boarini, San Francisco, CA (US)

(21) Appl. No.: 15/591,589

(22) Filed: May 10, 2017

Related U.S. Application Data
(60) Provisional application No. 62/334,290, filed on May 10, 2016.

(51) Int. Cl. G08B 21/04; A61B 5/024; A61B 5/00

(52) U.S. Cl. CPC G08B 21/0453; A61B 5/746; A61B 5/681; A61B 5/0022; A61B 5/02416; A61B 5/02438

(57) ABSTRACT

A life alert system which continually monitors a subject's heart rate via a wearable device such as a smart watch and automatically alerts call center personnel to engage emergency responders in the event of a cardiac incident, and provide the location of the subject. In response to measured physiological parameters, besides alerting the call center the watch attempts to communicate with the subject. After an emergency responder has been dispatched, the watch also displays medical information about the subject, providing an electronic “medic alert” bracelet function.

### CROSSREFERENCE TO RELATED APPLICATION

This patent application claims priority and benefit of U.S. Provisional Patent Application No. 62/334,290, entitled “Ibeat Autonomous Life Monitor System,” filed on May 10, 2016, the entire content of which is herein incorporated by reference for all purposes.

### FIELD OF THE INVENTION

The present invention is in the technical field of autonomous life monitors.

### BACKGROUND

The current state of life monitor technology consists of two or more devices including a base station and a radio receiver. Neither of these devices contains sensors monitoring the vitals of the user, with the exception of a few which provide an alert upon the user falling.

These mechanisms are generally tied to a hub or central station which does not allow the wearer to exit a defined range.

In addition, these systems are tied to either a conventional home telephone line or a remote connection via Bluetooth to a secondary device.

Lastly, action is required by the user to initiate a call for help so in the event of a user being incapacitated the system is rendered useless.

Therefore, a need exists for an autonomous device has unrestrained range, which passively monitors the user's vitals, and tracks his or her location to provide emergency care at the moment and location of incident.

### SUMMARY OF THE INVENTION

Embodiments of the disclosed invention are drawn to a system which is comprised of an autonomous, wrist-worn device that actively monitors the blood flow and pulse to detect whether certain cardiac thresholds have been crossed and which initiates emergency response protocols. Embodiments of the invention make use of local processes to detect the cardiac thresholds and cellular networks to directly connect to a web-based application that transmits the user's data to an emergency dispatch team.

More particularly, embodiments of the present invention include autonomous life monitors based on continual heart rate detection. More particularly, embodiments of the present invention are drawn to autonomous life monitors based on continual heart rate detection from a device worn on the wrist. More particularly, embodiments of the present invention are drawn to autonomous life monitors based on continual heart rate detection from a device worn on the wrist connected via cellular networks to a call center and EMT dispatch.

One embodiment of the invention includes a life monitor system in a watch form factor which continually monitors the wearer's heart rate and communicates with a call center in the event of a cardiac incident (defined as above or below specific heart rate thresholds).

The electronic system consists of, for example, (1) a digital “smart watch” worn around the wrist which contains a (1a) heart rate monitor via photoplethysmogram (PPG), (1b) a cellular antenna, (1c) a cellular module including SIM card, (1d) an accelerometer, (1e) vibration motor, (1f), telematics (GPS), (1g) the processor, (1h) a touchscreen interface, and (1i) battery. The watch system continually monitors the wearer's heart activity, e.g., heart rate, via PPG and detects when the wearer's pulse goes beyond pre-defined cardiac thresholds (e.g. below 20 bpm). If this occurs, the watch system will alert the wearer via vibration motor to confirm if they are having an incident via the touchscreen. If the wearer confirms an incident is taking place, the watch system will use the SIM card to place a call via cellular network(s) to communicate with the (2) Call Center, and provide the wearer's location (via GPS) and details of the emergency. The Call Center will (2a) dial the wearer's primary phone number and verify the emergency via (2b) Interactive Voice Response (IVR) and in the event of life-threatening emergency, (2c) dispatch EMT/emergency services based on the GPS of the watch. The Call Center will (2d) automatically notify pre-defined emergency contacts with details and location of the wearer, and (2e) manually contact emergency contacts when needed. All data from the watch are stored and accessible via (3) a SaaS web application and database. The database stores (3a) all heart activity (e.g., heart rate) data, (3b) wearer's emergency contacts' information, and (3c) wearer's medical history; all of which is accessible via a web portal (3d).

### BRIEF DESCRIPTION OF THE DRAWINGS

FIG. 1 is a summary of the emergency response computer controlled process which begins when a cardiac threshold is crossed.

FIG. 2 includes a number of watch system indicators including a view of the watch system during “usual activity” (e.g., cardiac threshold has not been crossed), a view of the watch system prompting for confirmation of an emergency once a cardiac threshold has been crossed, a view of the watch system asking if emergency services are required, a view of the watch system after emergency services have been dispatched remotely by the enterprise Call Center, a view of the watch system after family contacts have been notified of the incident and a view of the watch system during emergency mode displaying the wearer's allergies and medications.

FIG. 3 is a block diagram and data flow model of the database, segmentation, visual display, and manipulation by the end user.

FIG. 4 is a diagram of a general purpose computer system that can be used as a platform for the watch system in accordance with embodiments of the present invention.

### DETAILED DESCRIPTION

Reference will now be made in detail to the various embodiments of the present disclosure, examples of which are illustrated in the accompanying drawings. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

Some portions of the detailed descriptions that follow are presented in terms of procedures, logic blocks, processing, and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be a self-consistent sequence of steps or instructions leading to a desired result. The steps are those utilizing physical manipulations of physical quantities. Usually, although not necessarily, these quantities take the form of electrical or magnetic signals capable of being stored, transferred, combined, compared, and otherwise manipulated in a computer system. It has proven convenient at times, principally for reasons of common usage, to refer to these signals as transactions, bits, values, elements, symbols, characters, samples, pixels, or the like.

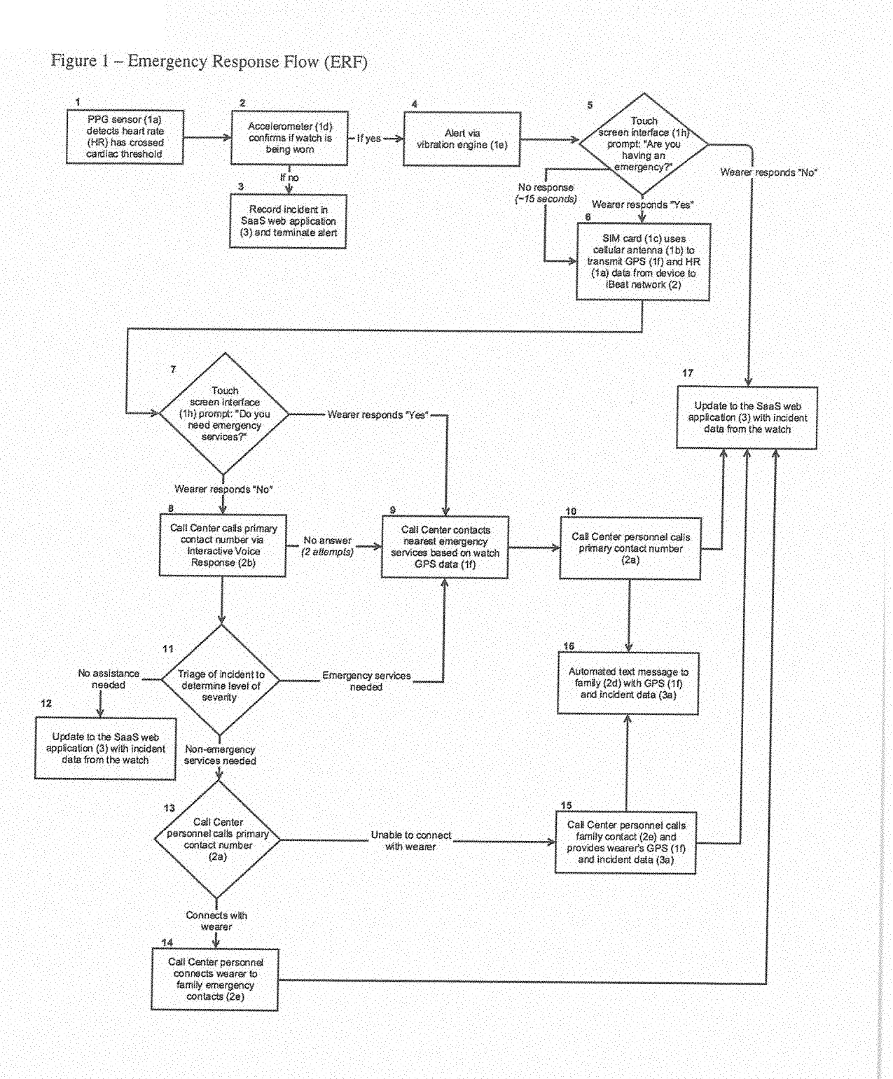

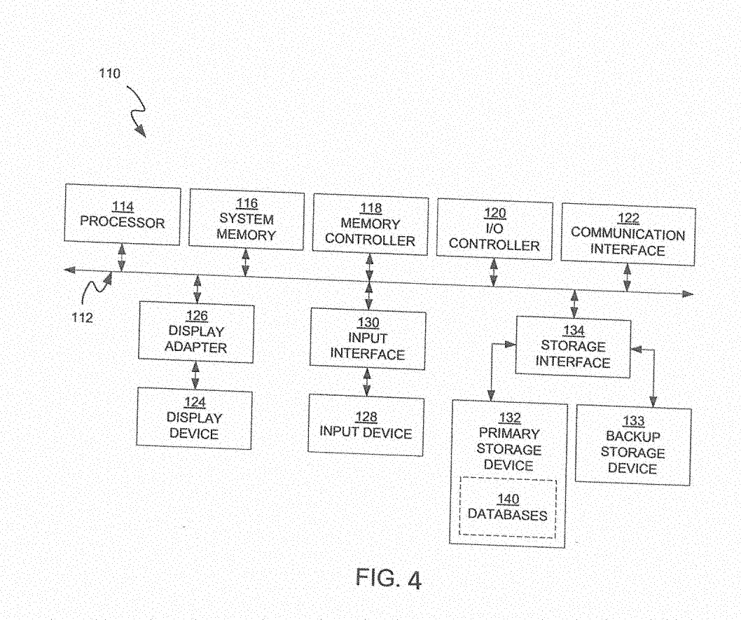

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “displaying,” “generating,” “producing,” “calculating,” “determining,” “radiating,” “emitting,” “attenuating,” “modulating,” “convolving,” “transmitting,” “performing,” or the like, refer to actions and processes (e.g., flowcharts of FIG. 1 and FIG. 3) of a computer system or similar electronic computing device or processor (e.g., system 110 of FIG. 4). The computer system or similar electronic computing device manipulates and transforms data represented as physical (electronic) quantities within the computer system memories, registers or other such information storage, transmission or display devices.

Embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer-readable storage media and communication media; non-transitory computer-readable media include all computer-readable media except for a transitory, propagating signal. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared, and other wireless media. Combinations of any of the above can also be included within the scope of computer-readable media.

Referring to the embodiments of the invention in greater detail, the electronic system consists of three components, which together create an emergency response flow (ERF): (1) a wearable, “smart watch” with a heart monitor via photoplethysmogram (PPG) and cellular connectivity; (2) a call center network and triage center; and (3) a web-based application to store, retrieve, and update wearer's information.

The wearer is able to add information for the watch system for emergency contacts, e.g. friends or family, via web portal (see FIG. 3, block 3.4). This contact information will be used in the event of an ERF to notify appropriate contacts.

The watch system has a unique Globally Unique Identifier (GUID) which assigns the physical device to the user's account which stores all relevant data; this removes the requirement of having any personally identifiable information (PPI) on the device itself—as well as removes the need to transmit any PPI via cellular network.

The watch system contains the PPG sensor (1a) which actively monitors the wearer's heart rate at all times by measuring the flow of the heart rate. The sensor is active all the time and is continually monitoring the wearer's heart rate.

This data from the PPG are collected and stored within the CPU (1g) on board the watch system. During usual activity (defined as the heart rate detected is in the “normal range,” e.g., 60-100 bpm), the data are stored locally on the device and uploaded asynchronously to the enterprise database (3a).

The event-triggering heart rate range, heretofore referred to as “cardiac threshold” is defined as, for example: (a) exceeding 85% the wearer's maximum heart rate, as determined by age and sex; (b) deceeding 30 bpm, irrespective of the wearer's age and sex. Of course, any other well known threshold could be used.

In the event that the PPG sensor detects a cardiac threshold has been crossed, multi-step emergency response flow (ERF) is initiated. Refer to FIG. 1 which illustrates a computer controlled process in accordance with embodiments of the present invention.

The first step in this process is to confirm the watch system is being worn via the PPG light sensor (see FIG. 1, step 2). If the watch system is not being worn, the ERF concludes with data sent to the enterprise database asynchronously (see FIG. 1, step 3).

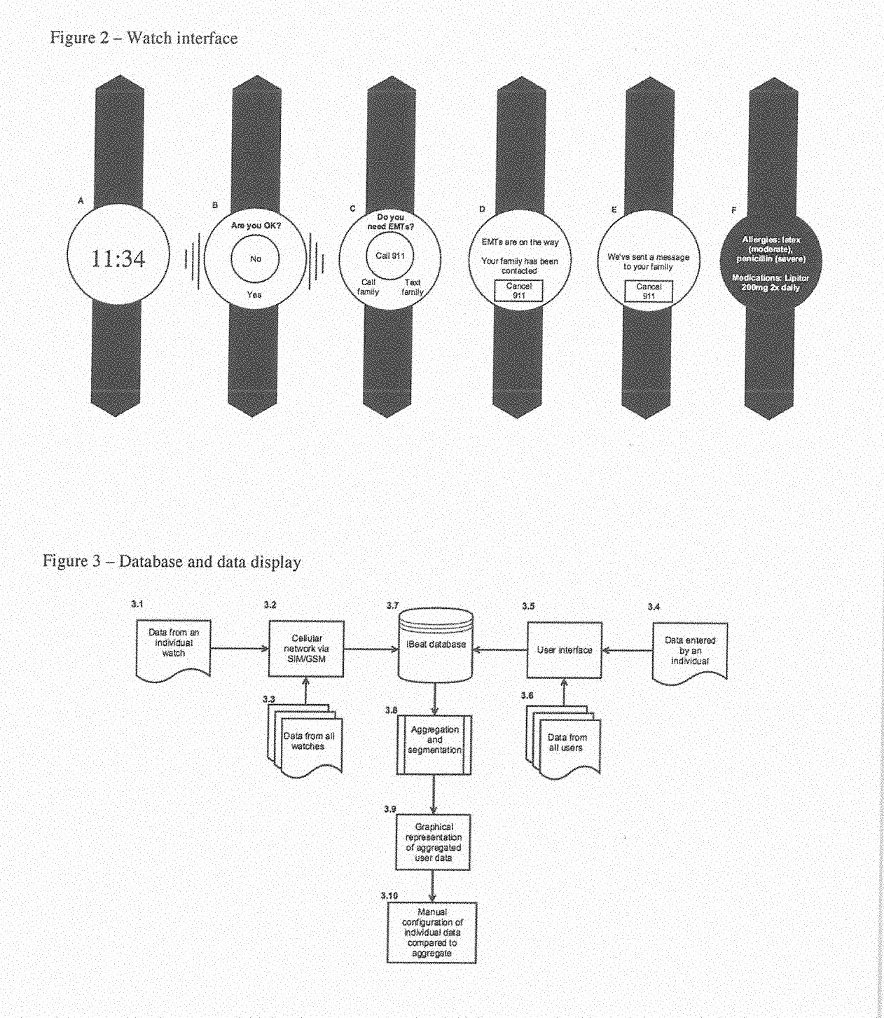

Following a confirmation that the watch is being worn, the watch system activates a vibration motor (see FIG. 1, step 4) to create a haptic alert on the wearer's wrist (FIG. 2, display B).

The haptic alert above is accompanied by the touch screen interface activating and presenting (displaying to) the user with a binary dialog or display queue to confirm there is an emergency (see FIG. 1, step 5), eg. “Are you having an emergency?” (see FIG. 2, display B).

The user is presented with two choices to confirm the emergency (see FIG. 2, display B). The user interfacing with a user interface device to select cancel or non-confirmation of emergency will terminate the ERF.

After a predetermined threshold of time, non response to this prompt (or any) will be considered a confirmation of emergency.

Once the emergency has been confirmed, the SIM card and cellular antenna will transmit the wearer's GUID, location, and heart rate data to the enterprise database.

Concurrently with the above, a prompt asks the wearer whether or not emergency services are required (see FIG. 1, step 7), as displayed in (see FIG. 2, display C).

If the wearer requires emergency services, a Call Center respondent will contact local 911 services, based on wearer's GPS coordinates at time of incident (see FIG. 1, step 9).

At this point, a message will notify the wearer that emergency services have been engaged (see FIG. 2, display D) and present the wearer with the option to cancel.

Following successful engagement of local 911, the call center will manually contact the wearer by their primary contact number (see FIG. 1, step 10).

Whether the call center is able to communicate directly with the wearer or not, the enterprise system will automatically trigger text messages to the wearer's pre-defined family contacts. (see FIG. 1, step 16) which contains the status of the ERF, the wearer's location via GPS, and any additional information.

The watch system will display the above information to the wearer to communicate that the wearer's family has been made aware of the situation (see FIG. 2, display E).

The watch system will display previously entered medical information, including allergies and medications, in the event the wearer is not able to communicate with emergency response personnel (see FIG. 2, display F).

The Call Center will remain in direct contact with the wearer until such time that emergency response personnel or family contacts are able to take over.

Once emergency response personnel or family contacts have reached the wearer, call center will consider the ERF complete and will mark the incident as closed, with all relevant data from the watch and Call Center stored in the database for future review (see FIG. 1, display 17).

If the wearer indicates emergency services are not required via the watch (see FIG. 2, display C), the Call Center will reach out to the wearer's primary contact number with interactive voice response (IVR) technology (see FIG. 1, step 8).

The IVR will triage the nature of the incident (see FIG. 1, step 9) to determine what level of response (if any) is required by the wearer.

If emergency services are determined to be required following the IVR triage, the ERF transmits the wearer's information and the Call Center engages with local 911, based on wearer's current GPS (see FIG. 1, step 9).

If no services of any kind are needed, the ERF is terminated and all relevant data is stored in the enterprise database for future review (see FIG. 1, step 12).

If non-emergency services are required, the Call Center personnel will directly contact the wearer via their primary contact information (see FIG. 1, step 13).

In the event the wearer cannot be reached, but the wearer has actively confirmed emergency services are not required, the Call Center personnel will directly call family contacts to provide all relevant data, including GPS and heart rate data (see FIG. 1, step 15).

This will be supplemented by automating text messages to other family contacts (see FIG. 1, step 16) and termination of the ERF, which will result in storage of all data (see FIG. 1, step 17).

If the wearer is reached directly by phone, the Call Center personnel will connect the wearer with family contacts, as needed (and determined by the wearer) (FIG. 1, 14).

Upon transfer of the wearer to family contacts, the ERF will be considered complete and the data will be stored in the enterprise database (FIG. 1, 17).

FIG. 3 is a block diagram and data flow model of the database, segmentation, visual display, and manipulation by the end user. Block 3.7 is a representation of the database which stores all data. Block 3.1 is a representation of data from an individual watch system 3.3. Block 3.2 is a representation of the cellular network upon which the data in 3.1 is carried. Block 3.3 is a representation of all watches in the network. Block 3.4 is a representation of an individual user's data. Block 3.5 is a representation of the user interface by which an individual enters data into the database. Block 3.6 is a representation of all data entered by users in the enterprise network. Block 3.8 is a representation of proprietary aggregation and segmentation of data collected above. Block 3.9 is a representation of graphical output of the data, following segmentation and aggregation. Block 3.10 is a representation of the manual control of various elements for segmentation and comparison.

With reference to FIG. 3, which shows an electronic system, the wearer's health data and activity collected by the watch or other sensor (Block 3.1), are transmitted via cellular network (Block 3.2) and stored in a database (Block 3.7). This includes heart rate, motion activity, temperature, oxygen saturation, perspiration, GPS, diagnostic data of the watch, and any other data available.

This process extends to all wearers on the network (Block 3.3).

In addition, the database houses all data entered manually by an individual wearer (Block 3.4) via a user interface (Block 3.5) within a secure web portal. This data includes any demographic data including: age, gender, location, marital status, or other data the enterprise may collect. In addition, any and all relevant medical information can be collected, including: medical conditions, medication history, doctor's information, or any other information the enterprise may collect.

This process is extended to all users on the network (Block 3.6).

The data is sorted, aggregated, summarized, analyzed, and any other function the enterprise determines necessary to report upon to its users (Block 3.8) including segmentation by age, gender, occupation, location, medical condition(s), current medication(s), marital status, or any other piece of information one can collect or infer from existing data.

The aggregated data can be displayed to individual users via interactive graph, table, chart or similar visual representation (Block 3.9). Individuals are able to review and compare their own data against any of the above criteria or any other data point.

Individuals are able to manipulate the aggregated and reported data in a visual display (Block 3.10) to compare against individuals within his or her similar demographics or any other data.

Within the web portal, a wearer is able to configure their contact information, as well as basic demographic data: age, sex, address.

The web portal includes workflows for the wearer to enter and update relevant medical information, including current medications (dose and frequency), allergies (including reaction), and diagnoses, and any other medical information that may be deemed relevant.

The above data can be presented on the display of the watch system during an emergency (see FIG. 2, display F) to provide emergency services or other related parties with critical medical data.

FIG. 4 is a block diagram of an example of a computing system 110 suitable for providing a hardware platform for the watch system of the present disclosure. Computing system 110 broadly represents any single or multi-processor computing device or general purpose computer system capable of executing computer-readable instructions, as shown in FIGS. 1 and 3, for instance. Examples of computing system 110 include, without limitation, distributed computing systems, handheld devices, worn devices (e.g. wrist-mounted watch system or waist-worn devices), or any other computing system or device. In its most basic configuration, computing system 110 may include at least one processor 114 and a system memory 116.

Processor 114 generally represents any type or form of processing unit capable of processing data or interpreting and executing instructions. In certain embodiments, processor 114 may receive instructions from a software application or module. These instructions may cause processor 114 to perform the functions of one or more of the example embodiments described and/or illustrated herein.

System memory 116 generally represents any type or form of volatile or non-volatile storage device or medium capable of storing data and/or other computer-readable instructions. Examples of system memory 116 include, without limitation, RAM, ROM, flash memory, or any other suitable memory device. Although not required, in certain embodiments computing system 110 may include both a volatile memory unit (such as, for example, system memory 116) and a non-volatile storage device (such as, for example, primary storage device 132).

Computing system 110 may also include one or more components or elements in addition to processor 114 and system memory 116. For example, in the embodiment of FIG. 4, computing system 110 includes a memory controller 118, an input/output (I/O) controller 120, and a communication interface 122, each of which may be interconnected via a communication infrastructure 112. Communication infrastructure 112 generally represents any type or form of infrastructure capable of facilitating communication between one or more components of a computing device. Examples of communication infrastructure 112 include, without limitation, a communication bus (such as an Industry Standard Architecture (ISA), Peripheral Component Interconnect (PCI), PCI Express (PCIe), or similar bus) and a network.

Memory controller 118 generally represents any type or form of device capable of handling memory or data or controlling communication between one or more components of computing system 110. For example, memory controller 118 may control communication between processor 114, system memory 116, and I/O controller 120 via communication infrastructure 112.

I/O controller 120 generally represents any type or form of module capable of coordinating and/or controlling the input and output functions of a computing device. For example, I/O controller 120 may control or facilitate transfer of data between one or more elements of computing system 110, such as processor 114, system memory 116, communication interface 122, display adapter 126, input interface 130, and storage interface 134.

Communication interface 122 broadly represents any type or form of communication device or adapter capable of facilitating communication between example computing system 110 and one or more additional devices. For example, communication interface 122 may facilitate communication between computing system 110 and a private or public network including additional computing systems and/or a call center. Examples of communication interface 122 include, without limitation, a wired network interface (such as a network interface card), a wireless network interface (such as a wireless network interface card), a modem, and any other suitable interface. In one embodiment, communication interface 122 provides a direct connection to a remote server via a direct link to a network, such as the Internet. Communication interface 122 may also indirectly provide such a connection through any other suitable connection.

Communication interface 122 may also represent a host adapter configured to facilitate communication between computing system 110 and one or more additional network or storage devices (or call center) via an external bus or communications channel. Examples of host adapters include, without limitation, Small Computer System Interface (SCSI) host adapters, Universal Serial Bus (USB) host adapters, IEEE (Institute of Electrical and Electronics Engineers) 1394 host adapters, Serial Advanced Technology Attachment (SATA) and External SATA (eSATA) host adapters, Advanced Technology Attachment (ATA) and Parallel ATA (PATA) host adapters, Fibre Channel interface adapters, Ethernet adapters, or the like. Communication interface 122 may also allow computing system 110 to engage in distributed or remote computing. For example, communication interface 122 may receive instructions from a remote device or send instructions to a remote device for execution.

As illustrated in FIG. 4, computing system 110 may also include at least one display device 124 coupled to communication infrastructure 112 via a display adapter 126. Display device 124 generally represents any type or form of device capable of visually displaying information forwarded by display adapter 126. Similarly, display adapter 126 generally represents any type or form of device configured to forward graphics, text, and other data for display on display device 124.

As illustrated in FIG. 4, computing system 110 may also include at least one input device 128 coupled to communication infrastructure 112 via an input interface 130. Input device 128 generally represents any type or form of input device capable of providing input, either computer- or human-generated, to computing system 110. Examples of input device 128 include, without limitation, a keyboard, a pointing device, a speech recognition device, an eye-track adjustment system, environmental motion-tracking sensor, an internal motion-tracking sensor, a gyroscopic sensor, accelerometer sensor, an electronic compass sensor, one or more physiological sensors, or any other input device.

As illustrated in FIG. 4, computing system 110 may also include a primary storage device 132 and a backup storage device 133 coupled to communication infrastructure 112 via a storage interface 134. Storage devices 132 and 133 generally represent any type or form of storage device or medium capable of storing data and/or other computer-readable instructions. For example, storage devices 132 and 133 may be a magnetic disk drive (e.g., a so-called hard drive), a flash drive, or the like. Storage interface 134 generally represents any type or form of interface or device for transferring data between storage devices 132 and 133 and other components of computing system 110.

In one example, databases 140 may be stored in primary storage device 132. Databases 140 may represent portions of a single database or computing device or it may represent multiple databases or computing devices. For example, databases 140 may represent (be stored on) a portion of computing system 110 and/or portions of example network architecture 200 in FIG. 2 (below). Alternatively, databases 140 may represent (be stored on) one or more physically separate devices capable of being accessed by a computing device, such as computing system 110 and/or portions of network architecture 200.

Continuing with reference to FIG. 4, storage devices 132 and 133 may be configured to read from and/or write to a removable storage unit configured to store computer software, data, or other computer-readable information. Examples of suitable removable storage units include, without limitation, a flash memory device, or the like. Storage devices 132 and 133 may also include other similar structures or devices for allowing computer software, data, or other computer-readable instructions to be loaded into computing system 110. For example, storage devices 132 and 133 may be configured to read and write software, data, or other computer-readable information. Storage devices 132 and 133 may also be a part of computing system 110 or may be separate devices accessed through other interface systems.

Many other devices or subsystems may be connected to computing system 110. Conversely, all of the components and devices illustrated in FIG. 4 need not be present to practice the embodiments described herein. The devices and subsystems referenced above may also be interconnected in different ways from that shown in FIG. 4. Computing system 110 may also employ any number of software, firmware, and/or hardware configurations. For example, the example embodiments disclosed herein may be encoded as a computer program (also referred to as computer software, software applications, computer-readable instructions, or computer control logic) on a computer-readable medium.

The computer-readable medium containing the computer program may be loaded into computing system 110. All or a portion of the computer program stored on the computer-readable medium may then be stored in system memory 116 and/or various portions of storage devices 132 and 133. When executed by processor 114, a computer program loaded into computing system 110 may cause processor 114 to perform and/or be a means for performing the functions of the example embodiments described and/or illustrated herein. Additionally or alternatively, the example embodiments described and/or illustrated herein may be implemented in firmware and/or hardware.

What is claimed is:

1. A method of providing assistance to a subject based on a determined physiological condition of the subject, the method operable between a wearable device and a call center, the method comprising:
based on information from one or more physiological sensors of the wearable device, determining that a physiological condition of the subject is abnormal;
transmitting an alert to the call center indicating the abnormal physiological condition of the subject and a determined location of the subject; and
determining any required emergency services required based on the abnormal condition and the determined location of the subject, and dispatching the emergency services.

2. The method of claim 1 further comprising responding to interaction between the wearable device and the subject.

3. The method of claim 2 further comprising displaying medic alert information on the wearable device after the interaction with the subject.

4. The method of claim 3 wherein the medic alert information comprises any one of current medications; allergies; and significant diagnoses or conditions.

5. The method of claim 2 wherein the transmitting an alert to the call center further comprises:
upon determining the abnormal condition, the wearable device prompting the subject and waiting for a response therefrom; and
if the subject does not respond to the prompt within a predetermined amount of time, the wearable device transmitting the alert to the call center.

6. The method of claim 1 wherein the dispatching the emergency services comprises determining emergency service providers nearest to the subject.

7. The method of claim 1 wherein the determining that a physiological condition of the subject is abnormal further comprises:
continually monitoring a physiological condition to determine a value for a normal condition; and
provided the physiological condition value varies from the normal condition more than a predetermined amount, generating the alert.

8. A method of providing assistance to a subject based on a determined physiological condition of the subject, the method operable between a wearable device and a call center, the method comprising:
based on information from one or more physiological sensors of the wearable device, determining that a physiological condition of the subject is abnormal; and
transmitting an alert to the call center indicating the abnormal physiological condition of the subject and a determined location of the subject, wherein said transmitting comprises:
upon determining the abnormal condition, the wearable device prompting the subject and waiting for a response therefrom; and
if the subject does not respond to the prompt within a predetermined amount of time, the wearable device transmitting the alert to the call center.

9. The method of claim 8 further comprising responding to interaction between the wearable device and the subject.

10. The method of claim 9 further comprising displaying medic alert information on the wearable device after the interaction with the subject.

11. The method of claim 9 wherein the medic alert information comprises any one of current medications; allergies; and significant diagnoses or conditions.

12. The method of claim 8 wherein the determining that a physiological condition of the subject is abnormal further comprises:
continually monitoring a physiological condition to determine a value for a normal condition; and
if the physiological condition value varies from the normal condition more than a predetermined amount, then generating an alert.

13. A watch system comprising a processor coupled to a bus, memory coupled to said bus, a display coupled to said bus, and one or more physiological sensors coupled to said bus, wherein said memory comprises instructions for implementing a method of providing assistance to a subject based on a determined physiological condition of the subject, the method operable between the watch system and a call center, the method comprising:
based on information from the one or more physiological sensors, determining that a physiological condition of the subject is abnormal; and
transmitting an alert for receipt by the call center indicating the abnormal physiological condition of the subject and a determined location of the subject, wherein said transmitting comprises:
upon determining the abnormal condition, prompting the subject and waiting for a response therefrom; and
if the subject does not respond to the prompt within a predetermined amount of time, transmitting the alert to the call center.

14. The system of claim 13 wherein the method further comprises responding to interaction between the watch system and the subject.

15. The system of claim 14 wherein the method further comprises displaying medic alert information on the display of the watch system after the interaction with the subject.

16. The system of claim 14 wherein the medic alert information comprises any one of current medications; allergies; and significant diagnoses or conditions.

17. The system of claim 13 wherein the determining that a physiological condition of the subject is abnormal further comprises:
continually monitoring a physiological condition to determine a value for a normal condition; and
if the physiological condition value varies from the normal condition more than a predetermined amount, then generating an alert.
